(19) United States
(12) Patent Application Publication
(10) Pub. No.: US 2025/0229010 A1
(43) Pub. Date: Jul. 17, 2025

(54) PRE-FILLED PLASTIC SYRINGE CONTAINING A VEGF ANTAGONIST

(71) Applicant: FORMYCON AG, Martinsried/Planegg (DE)

(72) Inventor: Bernd FIEDLER, Planegg (DE)

(73) Assignee: FORMYCON AG, Martinsried/Planegg (DE)

(21) Appl. No.: 19/169,549

(22) Filed: Apr. 3, 2025

Related U.S. Application Data
(63) Continuation of application No. 17/867,098, filed on Jul. 18, 2022.
Application No. 17/867,098 is a continuation of application No. 15/776,418, filed on May 16, 2018, now abandoned.

(30) Foreign Application Priority Data
Nov. 18, 2015 (EP) 15195177.9

(51) Int. Cl. A61L 31/04; A61K 39/395; A61M 5/00; A61M 5/28; A61M 5/31; A61M 5/315; A61M 5/32

(52) U.S. Cl. CPC A61L 31/048; A61K 39/3955; A61M 5/28; A61M 5/3129; A61M 5/31505; A61M 5/31513; A61M 5/3286; A61M 5/002; A61M 2005/3131

(57) ABSTRACT

The present invention relates to a pre-filled syringe containing a VEGF antagonist and comprising a plastic barrel which is silicone-free, kits comprising this syringe and the use of the syringe for the administration of a VEGF antagonist in the treatment of ocular diseases.

### FIELD OF THE INVENTION

The present invention relates to a pre-filled syringe containing a VEGF antagonist and comprising a plastic barrel which is silicone-free, kits comprising this syringe and the use of the syringe for the administration of a VEGF antagonist in the treatment of ocular diseases.

### BACKGROUND OF THE INVENTION

Ocular diseases such as age-related macular degeneration and diabetic macular edema are caused by the uncontrolled growth of blood vessels in the eye. Hence, one option to treat these and similar diseases is to inhibit angiogenesis in the eye. Since VEGF is a key factor in the stimulation of angiogenesis, it is an attractive target for down-regulating angiogenesis.

Aflibercept, marketed under the name Eylea®, is a recombinant fusion protein consisting of the VEGF binding portion from the extracellular domains of human VEGF receptors 1 and 2 that are fused to the Fc portion of the human IgG1 immunoglobulin. It is approved for the treatment of wet macular degeneration. Ranibizumab, marketed under the name Lucentis®, is a Fab fragment of a humanized murine monoclonal antibody directed against VEGF and has been approved for the treatment of ocular diseases such as age-related macular degeneration and diabetic macular edema. In addition, the off-label use of the full-length antibody bevacizumab (Avastin®) which is also directed against VEGF for the treatment of ocular diseases is common. Ranibizumab and bevacizumab appear to have similar efficacy profiles in the treatment of neovascular age-related macular degeneration although rare adverse events seem to occur more often with bevacizumab (Johnson and Sharma (2013) Curr. Opin. Ophthalmol.: 24(3): 205-12).

Both bevacizumab and ranibizumab are presented in glass vials from which they are usually drawn with a syringe shortly before injection into the eye. To use the whole content of the commercial vials of these antibodies, some companies repackage it in ready to use plastic syringes under sterile conditions, thereby allowing more than one syringe to be drawn from one glass vial. However, in the repackaged syringes silicone oil microdroplets and protein aggregates have been observed (Liu et al. (2011) Invest. Ophthalmol. Vis. Sci. 52(2): 1023-1034). Such silicone oil contaminants and protein aggregates may be responsible for the increase in intraocular pressure observed in patients treated with bevacizumab or ranibizumab (Kahook et al. (2009) Ophthalmic Surg. Lasers Imaging 40: 293-295; Good et al. (2011) Br. J. Ophthalmol. 95(8): 1111-1114).

AU 2012101677 A4 discloses pre-filled syringes containing a VEGF antagonist which syringes have a low silicone content. The whole disclosure of this document is focussed on the use of glass syringes and therefore teaches that a low amount of silicone has to be present within the syringe.

Further, recently a pre-filled ranibizumab syringe has been approved by the European Medicines Agency (EMA). The syringe barrel consists of borosilicate glass which was spray-coated with silicon oil-in-water emulsion and subsequently heat-fixed (so-called “baked silicone”) (poster presentation by Clunas et al. at the 5^{th} World Congress on Controversies in Ophthalmology, Mar. 20-23, 2014; poster presentation of Michaud et al. at the ARVO Annual Meeting 2014).

Pre-filled syringes have many benefits compared to a vial and a separately provided syringe, such as improved convenience, affordability, accuracy, sterility, and safety. The use of pre-filled syringes results in greater dose precision, in a reduction of the potential for needle sticks injuries that can occur while drawing medication from vials, in pre-measured dosage reducing dosing errors due to the need to reconstitute and/or draw medication into a syringe, and in less overfilling of the syringe helping to reduce costs by minimising drug waste.

However, glass syringes such as the approved ranibizumab pre-filled syringe are prone to breakage and have a relatively large weight compared to plastic syringes.

Further, they have to be treated with silicone to enable the correct movement of the stopper within the glass barrel and thereby effective and accurate drug delivery. It has been shown that silicone oil droplets occur in the vitreous cavity after intravitreal administration of VEGF antagonists and it was hypothesized that the silicone oil is derived from the needles and syringes used for the injections (Bakri and Ekdawi (2008) Retina 28: 996-1001).

Additionally, the glue which is necessary to attach a staked-in needle to a glass syringe can lead to impurities or increased protein oxidation (presentation of Adler at the 2011 PDA Europe The Universe of Pre-Filled Syringes and Injection Devices, Basel, 7-11 Nov. 2011; presentation of Markovic at the PDA Single Use Systems Workshop, Bethesda, 22-23 Jun. 2011).

Finally, during the manufacturing of glass pre-fillable syringes usually tungsten pins are used. It has been shown that soluble tungsten found in pre-filled syringes leads to protein aggregation and protein oxidation (Liu et al. (2010) PDA J. Pharm. Sci. Technol. 64(1): 11-19; Seidl et al. (2012) Pharm. Res. 29: 1454-1467).

Problems with glass pre-filled syringes have led to several product recalls in the past.

Several non-glass pre-filled syringes have been described. WO 2011/117878 A1 discloses a polycarbonate syringe, but it is not apparent whether the syringe barrel has been coated with silicone and whether the syringe is suitable for intraocular administration. WO 2009/099641 A2 discloses that in cyclic olefin polymer syringes without lubricant less visible particles form than in a glass syringe coated with silicone. However, it is not apparent whether this syringe can be used in ophthalmological applications.

Hence, there is still a need for non-glass syringes which can safely deliver the drug to the eye and which avoid the above disadvantages of using glass syringes, but in which the drug is stable for the storage period.

### SUMMARY OF THE INVENTION

The present inventors have surprisingly found that an anti-VEGF antibody solution is stable, i.e. the antibody is not significantly modified and does not aggregate significantly during storage when filled into a pre-filled syringe which comprises a silicone-free plastic syringe barrel, although it had been postulated that a plastic syringe is more permeable than a glass syringe for gases such as oxygen which may lead to protein modifications (see, e.g., Dierick and Yoshino (2015) OnDrugDelivery No. 55: 10-16). Hence, the syringe does not have to be packaged with an oxygen absorber. Further, the pre-filled syringe of the present invention does not contain a significant amount of particles. Finally, the forces required for injection of a solution from the pre-filled syringe of the present invention are comparable to the forces required for injection from a glass syringe.

The pre-filled syringe of the present invention therefore overcomes the disadvantages of glass syringes discussed above and may be used for administration of VEGF antagonists to the eye.

Accordingly, the present invention provides a pre-filled syringe containing a liquid formulation of a VEGF antagonist and comprising a syringe barrel, wherein the syringe barrel is made of plastic and is silicone-free, and further comprising a non-retractable stopper.

The present invention also relates to a pre-filled syringe containing a liquid formulation of a VEGF antagonist and comprising a syringe barrel, wherein the syringe barrel is made of plastic, is silicone-free and has a length of 45 mm to 65 mm.

In a preferred embodiment the VEGF antagonist is an anti-VEGF antibody or an antigen-binding fragment of such antibody or a soluble VEGF receptor fusion protein and more preferably the anti-VEGF antagonist is ranibizumab or aflibercept.

Preferably, the antagonist concentration is 1 to 100 mg/ml.

In one aspect of the invention the pre-filled syringe contains less than 50 particles per ml of the liquid formulation having a diameter of 10 μm or greater.

In another aspect of the invention the pre-filled syringe contains less than 5 particles per ml of the liquid formulation having a diameter of 25 μm or greater.

In still another aspect of the invention the pre-filled syringe has a gliding force of less than or equal to 10N.

In a preferred embodiment the pre-filled syringe further comprises a silicone-free stopper. More preferably, the stopper is coated with a fluoropolymer film.

Preferably, the syringe barrel is made of cycloolefin polymer or cycloolefin copolymer.

In a preferred embodiment the syringe barrel comprises an internal coating other than a silicone coating.

Also preferably, the pre-filled syringe comprises a staked needle.

The present invention also provides a kit comprising one or more pre-filled syringes according to the present invention. Preferably, the kit is a blister pack.

The pre-filled syringe may be used in administering a VEGF antagonist to a patient having an ocular disease, preferably having an ocular disease selected from the group consisting of age-related macular degeneration (AMD), visual impairment due to diabetic macular oedema (DME), visual impairment due to macular oedema secondary to retinal vein occlusion (branch RVO or central RVO), diabetic retinopathy in patients with diabetic macular edema or visual impairment due to choroidal neovascularisation (CNV) secondary to pathologic myopia.

Preferably, a volume of 30 to 100 μl of the liquid formulation is administered to the patient.

### BRIEF DESCRIPTION OF THE FIGURE

FIG. 1: Non-reduced SDS-PAGE analysis of the samples stored in the syringes S6 and S2 for three months at 40° C./75% relative humidity

### DETAILED DESCRIPTION OF THE INVENTION

The present invention as illustratively described in the following may suitably be practiced in the absence of any element or elements, limitation or limitations, not specifically disclosed herein.

The present invention will be described with respect to particular embodiments, but the invention is not limited thereto, but only by the claims.

Where the term “comprising” is used in the present description and claims, it does not exclude other elements. For the purposes of the present invention, the term “consisting of” is considered to be a preferred embodiment of the term “comprising”. If hereinafter a group is defined to comprise at least a certain number of embodiments, this is also to be understood to disclose a group which preferably consists only of these embodiments.

For the purposes of the present invention, the term “obtained” is considered to be a preferred embodiment of the term “obtainable”.

Where an indefinite or definite article is used when referring to a singular noun, e.g. “a”, “an” or “the”, this includes a plural of that noun unless something else is specifically stated.

A “pre-filled syringe” is a syringe which is supplied by the manufacturer in a filled state, i.e. a measured dose of the drug to be administered is already present in the syringe when it is purchased and ready for administration. In particular, the pharmaceutical composition containing the drug does not have to be drawn from a vial containing the composition by using an empty syringe. The term pre-filled syringe within the meaning of the present invention does not refer to syringes the content of which has been drawn from a vial in a repackaging process.

The drug contained in the pre-filled syringe of the present invention, i.e. the VEGF antagonist, preferably an anti-VEGF antibody, is stable at a temperature of 2 to 8° C. for at least six months, preferably for at least 9 months, more preferably for at least one year, particularly preferably for at least 18 months and most preferably for about two years. The drug contained in the pre-filled syringe of the present invention, i.e. the VEGF antagonist, preferably an anti-VEGF antibody or a VEGF receptor fusion protein and more preferably ranibizumab or aflibercept, is stable at room temperature, i.e. a temperature between 20° C. and 25° C., for at least three days or one week, preferably for at least two or three weeks, more preferably for about 4 weeks and most preferably for at least three months. The drug contained in the pre-filled syringe of the present invention, i.e. the VEGF antagonist, preferably an anti-VEGF antibody or a VEGF receptor fusion protein and more preferably ranibizumab or aflibercept, is stable at a temperature of about 40° C., for at least four or six hours, preferably for at least 10 or 12 hours, more preferably for at least 18 or 24 hours and most preferably for one or two weeks.

The stability of the drug within the syringe can for example be determined by ion exchange chromatography by which modifications of the drug such as oxidized and deamidated species can be detected or by size exclusion chromatography by which aggregates of the drugs can be detected. A description of such an analysis is provided in the examples section.

The drug, i.e. the VEGF antagonist, preferably the anti-VEGF antibody, is considered stable, if the sum of all impurities comprising aggregates and chemically modified species is less than 2%, preferably less than 1.5%, more preferably less than 1.2% and most preferably less than 1% compared to the amount of non-modified, non-aggregated drug.

The drug contained in the pre-filled syringe of the present invention, i.e. the VEGF antagonist, preferably an anti-VEGF antibody or a VEGF receptor fusion protein and more preferably ranibizumab or aflibercept, retains its biological activity when stored at a temperature of 2 to 8° C. for at least six months, preferably for at least 9 months, more preferably for at least one year, particularly preferably for at least 18 months and most preferably for about two years. The drug contained in the pre-filled syringe of the present invention, i.e. the VEGF antagonist, preferably an anti-VEGF antibody or a VEGF receptor fusion protein and more preferably ranibizumab or aflibercept, retains its biological activity when stored at room temperature, i.e. a temperature between 20° C. and 25° C. and 60% relative humidity for at least one day, preferably three days or one week, more preferably two weeks or three weeks and most preferably one month. The drug contained in the pre-filled syringe of the present invention, i.e. the VEGF antagonist, preferably an anti-VEGF antibody or a VEGF receptor fusion protein and more preferably ranibizumab or aflibercept, retains its biological activity when stored at a temperature of about 40° C. and 75% relative humidity for at least 1 hour or 2 hours, preferably for at least four or six hours, more preferably for at least 10 or 12 hours, and most preferably for at least 18 or 24 hours.

The biological activity of the VEGF antagonist, preferably an anti-VEGF antibody or a VEGF receptor fusion protein and more preferably ranibizumab or aflibercept can be determined by incubating different dilutions of the antagonist which was stored under the conditions described above with human umbilical vein endothelial cells (HUVEC) and VEGF and measuring the VEGF-induced proliferation of the cells in the presence of the antagonist, i.e. by the CellTiter-Blue® Cell Viability Assay available from Promega, in comparison to cells not incubated with the antagonist. Since the VEGF antagonist inhibits VEGF-induced signal transduction, the VEGF-induced proliferation will be reduced, if biologically active VEGF antagonist is present in the sample.

The VEGF antagonist, preferably the anti-VEGF antibody or VEGF receptor fusion protein and more preferably ranibizumab or aflibercept retains its biological activity after storage in the pre-filled syringe, such that the VEGF-induced proliferation is inhibited in HUVEC. The VEGF-antagonist, preferably the anti-VEGF antibody or VEGF receptor fusion protein and more preferably ranibizumab or aflibercept retains its biological activity after storage in the pre-filled syringe, if the VEGF-induced proliferation is inhibited by at least 50%, preferably by at least 55% or 60%, more preferably by at least 65%, 70%, 75% or 80%, even more preferably by at least 85%, 87% or 90% and most preferably by at least 92%, 94%, 96%, 98% or 99%.

The components of a pre-filled syringe are known to a skilled person and basically comprise, from the outlet to the rear end, a tip cap or needle shield, a syringe barrel, a stopper located within the syringe barrel and a plunger rod.

The syringe barrel contains a defined volume of the liquid composition which can be expelled from the barrel through an outlet positioned on one end of the barrel when the plunger rod is pushed into and moves along the barrel. The syringe barrel typically has a substantially cylindrical shape. The outlet may comprise a projection from the outlet end through which extends a channel having a smaller diameter than the rest of the syringe barrel. The outlet may be adapted, for example by a luer lock type connection, for connection with a needle if no staked needle is used. In this case, a tip cap is used to seal the barrel which can be removed to allow a needle to be attached to the syringe. This sealing can be achieved by the use of known sealing devices such as the OVS™ system of Vetter Pharma International GmbH. The tip cap is usually made of an elastomer which may comprise a fluoropolymer coating in the interior part which is in contact with the syringe.

In the pre-filled syringe of the present invention the syringe outlet may be firmly connected with a needle so that the pre-filled syringe is supplied with a staked needle and does not need to be assembled prior to use. In this case, the risk of injuries with the needle during assembly of the syringe before injection is reduced. Prior to use the staked needle is typically covered by a needle shield to ensure sterility of the syringe content.

The staked needle can be attached to the pre-filled plastic syringe of the present invention without using an adhesive, since it can be moulded into the syringe. In contrast, an adhesive is required to attach the needle to a glass syringe and can lead to impurities or increased protein oxidation (presentation of Adler at the 2011 PDA Europe “The Universe of Pre-Filled Syringes and Injection Devices”, Basel, 7-11 Nov. 2011; presentation of Markovic at the PDA Single Use Systems Workshop, Bethesda, 22-23 Jun. 2011).

For intravitreal administration the needle size is typically 29, 29½ or 30 gauge, although 31-, 32, 33- and 34-gauge needles may also be used. The pre-filled syringe may be equipped with a passive needle safety guard to further avoid the danger of needle sticks after injection.

The pre-filled syringe of the present invention comprises a syringe barrel which is made from plastic material. Preferably, the plastic material is selected from cycloolefin polymer and cycloolefin copolymer and more preferably it is a cycloolefin polymer and most preferably it is a cycloolefin polymer known as Crystal Zenith®.

Cycloolefin copolymers may be produced by chain copolymerization of cyclic monomers such as 8,9,10-trinomom-2-ene or 1,2,3,4,4a,5,8,8a-octahydro-1,4:5,8-dimethanonaphthalene with ethane. Suitable copolymers are those of the Topas™ type which are available in a variety of grades.

Cycloolefin polymers may for example be produced by ring-opening metathesis polymerization of various cyclic monomers followed by hydrogenation. Suitable commercially available containers made of cycloolefin polymer material include containers manufactured from Crystal Zenith™ resin, Zeonor™ and Zeonex™. Such materials have a glass-like transparency, are highly break resistant and provide an excellent moisture barrier.

According to the present invention the syringe barrel is silicone-free which means that the inner surface of the syringe barrel has not been treated with silicone oil. Hence, no silicone oil can be detected within the pre-filled syringe of the present invention.

The presence and thickness of silicone layers can be determined by known methods such as the rap.ID Layer Explorer® application which can also be used to measure the amount of silicone oil within the syringe barrel. The amount of silicone oil within the syringe barrel can also be measured by differential weighing methods and quantitation by infrared spectroscopy of the oil diluted in a suitable solvent.

The pre-filled syringe may be uncoated, i.e. the cycloolefin polymer or copolymer material is in direct contact with the liquid composition contained therein and the syringe barrel does not contain any material other than the plastic material of which the syringe is made.

Alternatively, the pre-filled syringe may comprise an internal coating other than a silicone coating. The term “internal coating” is intended to mean a coating on the inner side of the syringe barrel which is in contact with the drug solution, i.e. the liquid composition. Examples of such an internal coating include a fluorocarbon film made from a modified ethylene-tetrafluoroethylene copolymer (also called Flurotec® film, available from West Pharmaceutical Services) and a perfluoropolyether film crosslinked by an Atmospheric Plasma Immobilization™ process (also called TriboGlide®, available from TriboFilm Research and described in WO 2005/094214 A2).

Preferably, the pre-filled syringe does not comprise an internal coating.

The syringe may also comprise a coating on the outer surface of the syringe which is in contact with the environment such as an oxygen barrier coating.

The syringe barrel is tungsten-free, i.e. it does not contain any traces of tungsten, since it is not necessary to use tungsten in the syringe manufacturing process. Hence, there is no risk of tungsten-induced protein aggregation.

In one embodiment the syringe barrel comprises a mark such as a line printed on the syringe barrel which line allows the person injecting the liquid composition to align a pre-determined part of the stopper (such as the tip of the front surface) or plunger rod with the mark. Thereby, any excess liquid composition and potential air bubbles are removed from the syringe barrel, allowing the safe administration of an exact predetermined dosage to the patient.

The syringe barrel has a length of 45 to 65 mm. If the syringe has a nominal maximum fill volume of 1 ml, the length of the syringe barrel is 60 to 65 mm. If the syringe has a nominal maximum fill volume of 0.5 ml, the length of the syringe barrel is 45 to 50 mm. The length of the syringe barrel is the length between the rear end to the outlet to which the needle is attached (but not including the needle, if present).

The syringe barrel has an internal diameter of 4 to 6.5 mm. If the syringe has a nominal maximum fill volume of 1 ml, the internal diameter of the syringe barrel is 5.5 to 6.5 mm. If the syringe has a nominal maximum fill volume of 0.5 ml, the internal diameter of the syringe barrel is 4 to 5 mm.

The wall of the syringe barrel has a thickness of 0.6 to 1.2 mm, preferably of 0.8 to 1 mm and more preferably of 0.9 mm.

The plunger rod is pulled and pushed along inside the syringe barrel, allowing the syringe to expel the liquid formulation through the outlet. The plunger rod comprises a stopper contact surface, a rod and a flange (arranged from the outlet end to the rear end). When the plunger rod is moved through the syringe barrel from the rear part towards the outlet by applying pressure to the flange, the stopper contact surface of the plunger rod comes into contact with the rear part of the stopper and moves the stopper through the barrel to expel the liquid composition contained within the syringe through the outlet of the syringe barrel. The stopper contact surface of the plunger rod is preferably substantially flat, i.e. it does not comprise any protrusions for connection to the stopper.

The stopper is located within the syringe barrel between the syringe outlet and the plunger rod. The stopper is typically made of an elastomeric material such as natural or synthetic rubber, which engages an inner surface of the syringe barrel to create a seal that facilitates ejecting the liquid formulation from the syringe when pressure is applied to the flange of the plunger rod and the stopper moves through the syringe barrel. Since the stopper is not mechanically connected to the plunger rod before administration, it is not retractable. The term “non-retractable stopper” therefore is intended to mean that the stopper can only be moved in the direction of the syringe outlet, but not in the opposite direction, i.e. to the rear part of the syringe. It also means that the stopper and the plunger rod are not mechanically connected. Hence, any risk for the contamination of the liquid composition within the syringe is minimized.

The stopper may be coated with a fluoropolymer film such as an ethylene tetrafluoroethylene (ETFE; marketed as FluroTec®) barrier film, a fluorinated ethylene propylene (FEP; marketed as Teflon® FEP) or a polytetrafluoroethylene-like film such as used for an Omniflex stopper at least in that part which comes into contact with the liquid composition contained within the prefilled syringe. This type of coating serves as an effective barrier between the drug and the elastomer, reducing the potential for extractables or leachables which are inherent to all materials. In addition, the coating reduces the occurrence of the reverse process, where the drug product can adsorb or absorb into the plunger rod. The stopper is preferably silicone-free, i.e. at least the surface of the stopper which comes into contact with the drug solution and more preferably the complete stopper has not been coated with silicone oil. More preferably, the stopper is silicone-free and comprises a coating with ethylene tetrafluoroethylene.

The syringe has a nominal maximum fill volume, i.e. a volume which can be maximally taken up by the syringe, of 0.3 ml to 1.5 ml, preferably of 0.5 ml to 1.0 ml, more preferably of 0.5 ml or 1.0 ml and most preferably of 1.0 ml.

The volume of the liquid composition filled into the syringe is about 0.05 ml to about 1 ml, preferably about 0.1 ml to about 0.5 ml, more preferably 0.14 ml to 0.3 ml and most preferably 0.15 ml to 0.2 ml.

The skilled person knows that the syringe is usually filled with a volume which is larger than the volume actually administered to the patient to take into account any dead space within the syringe and the needle and the loss due to the preparation of the syringe for injection. Hence, the volume which is actually administered to the patient is between 0.01 ml and 1 ml, preferably between 0.02 and 0.5 ml, more preferably between 0.025 and 0.5 ml, even more preferably between 0.03 ml and 0.05 ml and most preferably the volume which is actually administered to the patient is 0.05 ml.

Ranibizumab is typically administered in a volume of 0.05 ml with a ranibizumab concentration of 6 or 10 mg/ml or in a volume of 0.03 ml or 0.05 ml with a ranibizumab concentration of 10 mg/ml, yielding a delivered amount of 0.3 or 0.5 mg. For aflibercept the administered volume is typically 0.05 ml with an aflibercept concentration of 40 mg/ml, yielding a delivered amount of 2 mg. As discussed above, bevacizumab is used off-label for the treatment of ocular diseases. In this case, the administered volume of bevacizumab is 0.05 ml with a bevacizumab concentration of 25 mg/ml, yielding a delivered amount of 1.25 mg.

Hence, in one embodiment the syringe is filled with a volume of the liquid composition of 0.15 ml to 0.2 ml and 0.03 ml to 0.05 ml of the liquid composition are then administered to the patient.

In a particular embodiment the pre-filled syringe of the present invention contains a liquid formulation of ranibizumab and comprises a silicone-free cycloolefin polymer syringe barrel, a tip cap or needle shield, a non-retractable silicone-free stopper and a plunger rod, wherein the stopper is coated with a fluoropolymer film.

In another particular embodiment the pre-filled syringe of the present invention contains a liquid formulation of ranibizumab and comprises a silicone-free cycloolefin polymer syringe barrel, a tip cap or needle shield, a stopper and a plunger rod, wherein the stopper is coated with a fluoropolymer film and wherein the syringe barrel has a length of 45 mm to 65 mm.

The term “VEGF antagonist” refers to a molecule which specifically interacts with VEGF and inhibits one or more of its biological activities, e.g. its mitogenic, angiogenic and/or vascular permeability activity. It is intended to include both anti-VEGF antibodies and antigen-binding fragments thereof and non-antibody VEGF antagonists.

Non-antibody VEGF antagonists include aflibercept, pegaptanib and antibody mimetics. Preferably, the non-antibody VEGF antagonist is aflibercept. Aflibercept which is presently marketed under the name Eylea® and which is also known as VEGF-trap is a recombinant human soluble VEGF receptor fusion protein in which portions of human VEGF receptors 1 and 2 extracellular domains are fused to the Fc portion of human IgG1 (Holash et al. (2002) Proc. Natl. Acad. Sci. USA 99(17): 11393-11398; WO 00/75319 A1). The CAS number of aflibercept is 862111-32-8. It has received a marketing authorization for the treatment of wet age-related macular degeneration, visual impairment due to diabetic macular oedema (DME) and diabetic retinopathy in patients with diabetic macular edema. The present commercial aflibercept formulation contains sodium phosphate, sodium chloride, polysorbate 20, sucrose and water for injection and is supplied in a concentration of 40 mg/ml. In particular, it contains 40 mg/ml Aflibercept, 10 mM sodium phosphate buffer, 40 mM NaCl, 0.03% polysorbate 20, 5% sucrose; and water for injection. An alternative aflibercept formulation may contain a histidine buffer, sodium chloride, polysorbate 20, sucrose and water for injection and is supplied in a concentration of 40 mg/ml. In particular, it contains 40 mg/ml Aflibercept, 10 mM histidine buffer, 40 mM NaCl, 0.03% polysorbate 20, 5% sucrose; and water for injection. The pH of the commercial and the alternative Aflibercept formulation may be adjusted to 6.2.

Pegaptanib which is presently marketed under the name Macugen® is a pegylated anti-vascular endothelial growth factor (VEGF) aptamer (Bell et al. (1999) In Vitro Cell Dev Biol Anim. 35(9): 533-42). The CAS number of pegaptanib is 222716-86-1.

Antibody mimetics which are VEGF antagonists include binding proteins comprising an ankyrin repeat domain that binds VEGF and inhibits its binding to the receptor, such as DARPin® MPO112 (see also WO 2010/060748 and WO 2011/135067).

The term “anti-VEGF antibody” refers to an antibody or antibody fragment such as a Fab or a scFV fragment that specifically binds to VEGF and inhibits one or more of its biological activities, e.g. its mitogenic, angiogenic and/or vascular permeability activity. Anti-VEGF antibodies act, e.g., by interfering with the binding of VEGF to a cellular receptor, by interfering with vascular endothelial cell activation after VEGF binding to a cellular receptor, or by killing cells activated by VEGF. Anti-VEGF antibodies include, e.g., antibodies A4.6.1, bevacizumab, ranibizumab, G6, B20, 2C3, and others as described in, for example, WO 98/45331, US 2003/0190317, U.S. Pat. Nos. 6,582,959, 6,703,020, WO 98/45332, WO 96/30046, WO 94/10202, WO 2005/044853, EP 0 666 868 BI, WO 2009/155724 and Popkov et al. (2004) J. Immunol. Meth. 288: 149-64. Preferably, the anti-VEGF antibody or antigen-binding fragment thereof present in the pharmaceutical composition of the present invention is ranibizumab or bevacizumab. Most preferably, it is ranibizumab or an antigen-binding fragment thereof.

“Ranibizumab” is a humanised monoclonal Fab fragment directed against VEGF-A having the light and heavy chain variable domain sequences of Y0317 as described in SEQ ID Nos. 115 and 116 of WO 98/45331 and Chen et al. (1999) J. Mol. Biol. 293: 865-81. The CAS number of ranibizumab is 347396-82-1. Ranibizumab inhibits endothelial cell proliferation and neovascularisation and has been approved for the treatment of neovascular (wet) age-related macular degeneration (AMD), the treatment of visual impairment due to diabetic macular oedema (DME), the treatment of visual impairment due to macular oedema secondary to retinal vein occlusion (branch RVO or central RVO), or treatment of visual impairment due to choroidal neovascularisation (CNV) secondary to pathologic myopia. Ranibizumab is related to bevacizumab and derived from the same parent mouse antibody as bevacizumab but it is much smaller than the parent molecule and has been affinity matured to provide stronger binding to VEGF-A. Ranibizumab is produced recombinantly in Escherichia coli, e.g. as described in WO 98/45331 A2. The present commercial ranibizumab formulation contains α,α-trehalose dihydrate, histidine hydrochloride monohydrate, histidine, polysorbate 20 and water for injection and is supplied in a concentration of 10 mg/ml. In particular, it contains 6 or 10 mg. Ranibizumab, 100 mg. α,α-trehalose dehydrate; 0.32 mg. L-histidine, 1.66 mg. L-histidine hydrochloride monohydrate, 0.1 mg Polysorbate 20 and water for injection qs to 1 mL. The pH of the present commercial Ranibizumab formulation may be adjusted to pH 5.5.

“Bevacizumab” is a full-length, humanized murine monoclonal antibody that recognizes all isoforms of VEGF and which is the parent antibody of ranibizumab. The CAS number of bevacizumab is 216974-75-3. Bevacizumab inhibits angiogenesis and is presently approved for the treatment of different cancer types. However, it is also used off-label in ophthalmological diseases such as age-related macular degeneration. The present commercial bevacizumab formulation contains α,α-trehalose dihydrate, sodium phosphate, polysorbate 20 and water for injection and is supplied as a concentrate with a concentration of 25 mg/ml. In particular, it contains 25 mg/ml Bevacizumab, 240 mg α,α-trehalose dihydrate, 23.2 mg sodium phosphate (monobasic, monohydrate), 4.8 mg sodium phosphate (dibasic, anhydrous), 1.6 mg polysorbate 20, and water for Injection, USP to 4 ml.

The antibody concentration within the pre-filled syringes of the present invention is typically 1-100 mg/ml, preferably 2-75 mg/ml, more preferably 3-50 mg/ml, even more preferably 5 to 30 mg/ml and most preferably 6 or 10 mg/ml. If ranibizumab is contained within the pre-filled syringe of the present invention the ranibizumab concentration is 10 mg/ml. If aflibercept is contained within the pre-filled syringe of the present invention the aflibercept concentration is 40 mg/ml.

The pre-filled syringe may contain one or more pharmacologically active agents in addition to the VEGF antagonist. A pharmacologically active agent is able to exert a pharmacological effect when administered to a subject. Preferably, the additional pharmacologically active agent is a PDGF antagonist or an Ang2 antagonist. More preferably, the PDGF antagonist is an anti-PDGF antibody such as rinucumab or an aptamer such as E10030, marketed as Fovista®. Most preferably, the PDGF antagonist is E10030 which is described in Green et al. (1996) Biochemistry 35: 14413; U.S. Pat. Nos. 6,207,816; 5,731,144; 5,731,424; and 6,124,449. Also more preferably, the Ang2 antibody is an anti-Ang2 antibody and most preferably it is nesvacumab.

The liquid composition within the pre-filled syringe of the present invention has a low particle content. In particular, it comprises less than 50 particles having a size of more than 10 μm after storage of the syringe at 5° C. or 25° C. or 40° C. for three months. Alternatively or additionally, it comprises less than 5 particles having a size of more than 25 μm after after storage of the syringe at 5° C. or 25° C. or 40° C. for three months. Hence, the pre-filled syringe meets the requirements of United States Pharmacopoiea <789> for ophthalmic solutions with respect to these particle sizes.

The pre-filled syringe of the present invention further has excellent gliding behaviour. In particular, the break loose force, i.e. the force required to initiate the movement of the plunger rod, is less than 10N or 9N, preferably less than 8N or 7N, more preferably less than 6N and most preferably less than 5N. The break loose force does not change significantly, i.e. it is still within the ranges specified above, when the syringe is stored for an extended period such as one or three months at a temperature of 5° C., 25° C. or 40° C. In contrast, in a syringe containing silicone the break loose force shows a stronger increase upon storage.

Further, the gliding force, i.e. the force required to sustain the movement of the plunger along the syringe barrel to expel the liquid composition, is less than 10N, preferably less than 9N, more preferably less than 8N, even more preferably less than 7N and most preferably less than 6N. The gliding force does not change significantly, i.e. it is still within the ranges specified above, when the syringe is stored for an extended period such as one or three months at a temperature of 5° C., 25° C. or 40° C.

The present invention also provides a kit comprising one or more of the pre-filled syringes of the present invention. Preferably, the kit comprises a blister pack. A “blister pack” has a cavity or pocket which is usually made from thermoformed plastic and a backing of paperboard or a lidding seal of aluminium foil or plastic. The blister pack may be sterilized before the sterile syringe is packaged into it under aseptic conditions. Hence, no sterilization after packaging is required. The kit may further comprise a needle, if the pre-filled syringe does not comprise a staked-in needle. The kit may further comprise instructions for use.

Preferably, the kit does not comprise an oxygen absorber which is typically used to reduce the level of oxygen within a package such as a blister pack. Oxygen absorbers usually contain a substance such as ferrous carbonate or ascorbate which substance reacts with any oxygen within a package with a high affinity, thereby reducing the oxygen content of the package.

An “intraocular neovascular disease” is a disease characterized by ocular neovascularisation. Examples of intraocular neovascular diseases include, e.g., proliferative retinopathies, choroidal neovascularisation (CNV), age-related macular degeneration (AMD), diabetic and other ischemia-related retinopathies, diabetic macular oedema, diabetic retinopathy in patients with diabetic macular edema, pathological myopia, von Hippel-Lindau disease, histoplasmosis of the eye, Central Retinal Vein Occlusion (CRVO), Branch Retinal Vein Occlusion (BRVO), corneal neovascularisation, and retinal neovascularisation. The term “age-related macular degeneration” refers to a medical condition which usually affects older adults and results in a loss of vision in the centre of the visual field (the macula) because of damage to the retina.

Preferably, the pre-filled syringe is for use in the intravitreal injection of a VEGF antagonist as defined herein.

The term “intravitreal injection” refers to the administration of a pharmaceutical composition in which the substance is injected directly into the eye. More specifically, the substance is injected into the vitreous humour (also called vitreous body or simply vitreous) which is the clear gel that fills the space between the lens and the retina of the eyeball of humans and other vertebrates.

While the invention has been illustrated and described in detail in the drawings and foregoing description, such illustration and description are to be considered illustrative or exemplary and not restrictive. The invention is not limited to the disclosed embodiments. Other variations to the disclosed embodiments can be understood and effected by those skilled in the art in practicing a claimed invention, from a study of the drawings, the disclosure, and the dependent claims.

The detailed description is merely exemplary in nature and is not intended to limit application and uses. The following examples further illustrate the present invention without, however, limiting the scope of the invention thereto. Various changes and modifications can be made by those skilled in the art on the basis of the description of the invention, and such changes and modifications are also included in the present invention.

### Examples

### 1. Determination of Particles of Different Sizes in Different Syringes and Subjected to Different Conditions

400 μl of a solution containing histidine buffer, trehalose dihydrate, polysorbate 20, pH 5.5, i.e. the components of the ranibizumab formulation, but not ranibizumab itself, was filled into the following syringes:

TABLE 1

| | Syringe | Syringe | Syringe | Silicone level | |
|---|---|---|---|---|---|
| No. | size | barrel | type | [mg] | Stopper coating |
| 2 | 1.0 ml | Borosilicate | Luer cone | 0.16 (baked-on) | Fluoropolymer |
| | | glass | | | (Flurotec) |
| 3 | 1.0 ml | Borosilicate | Luer cone | 0.7 (baked-on) | Fluoropolymer |
| | | glass | | | (Flurotec) |
| 4 | 1.0 ml | Borosilicate | Staked | 0.25 ± 0.2 | Fluoropolymer |
| | | glass | needle | | (Flurotec) |
| 5 | 1.0 mL | Cycloolefin | Luer cone | 1.5 | Cross-linked |
| | | polymer | | | silicone |
| 6 | 1.0 mL | Cycloolefin | Luer cone | No silicone | Fluoropolymer |
| | | polymer | | | (Flurotec) |

The syringes from Table 1 were incubated at 5° C., 25° C./60% relative humidity and 40° C./75% relative humidity for three months. Afterwards, the light obscuration was determined with the FlowCam PV bench top system (Fluid Imaging Technologies Inc., Maine, USA) using the System software (VisualSpreadsheet software, version 3.4.8) and the following parameters:

| | |
|---|---|
| Mode: | AutoImage |
| Priming Method: | manual prime with sample |
| Flow Rate: | 0.100 ml/min |
| Recalibrations: | 0 |
| Stop Reason: | Sample Volume |
| Sample Volume Aspirated: | 1.0421 ml |
| Sample Volume Processed: | 1.0392 ml |
| Fluid Volume Imaged: | 0.3421 ml |
| Frame Rate: | 22.00 fps |
| Magnification: | 10X |
| Calibration Factor: | 0.6979 |
| Syringe Size: | 1.00 ml |

The results of the analysis are shown in Table 2. The pre-filled silicone-free cycloolefin polymer syringe 6 had low particle levels under all conditions tested.

TABLE 2

| | | Particle count/mL [mean] | | |
|---|---|---|---|---|
| | | | | 1-500 |
| Condition | Syringe | ≥10 μm | ≥25 μm | μm |
| | Buffer (mean of | 11 | 1 | 50 |
| | 22 measurements) | | | |
| 3 M 5° C. | S2 | 10 | 1 | 60 |
| | S3 | 47 | 2 | 278 |
| | S4 | 276 | 3 | 1066 |
| | S5 | 195 | 2 | 1138 |
| | S6 | 13 | 2 | 137 |
| 3 M 25° C./60% r.H. | S2 | 11 | 0 | 101 |
| | S3 | 27 | 0 | 437 |
| | S4 | 1518 | 41 | 3831 |
| | S5 | 124 | 4 | 529 |
| | S6 | 23 | 2 | 128 |
| 3 M 40° C./75% r.H. | S2 | 8 | 0 | 88 |
| | S3 | 8 | 0 | 67 |
| | S4 | 2316 | 37 | 6121 |
| | S5 | 172 | 12 | 669 |
| | S6 | 22 | 3 | 349 |

M: months

### 2. Determination of Ranibizumab Stability in Plastic and Glass Syringes Subjected to Different Conditions

165 μl of a solution containing 10 mg/ml of the anti-VEGF antibody ranibizumab and histidine buffer, trehalose dihydrate, polysorbate 20, pH 5.5 was filled into the syringes as listed above in Table 1.

The syringes as listed above in Table 1 were incubated at 25° C./60% relative humidity and 40° C./75% relative humidity for two weeks, one month and three months and at 5° C. for three months.

Afterwards, the samples were analyzed by RP-HPLC for the presence of hydrophilic species, by cation exchange chromatography for the presence of acidic and basic variants of the antibody and by size exclusion chromatography for the presence of aggregates.

### a) RP-HPLC Analysis

The protein samples from the syringes were loaded onto a ZORBAX 300SB-C18, 4.6×100 mm, 3.5 μm column to detect hydrophilic and hydrophobic impurities.

The protein was eluted with a gradient of eluent A (0.1% trifluoroacetic acid in water) and eluent B (0.1% trifluoroacetic acid in 70% acetonitrile, 20% 1-propanol and 10% water) according to the following Table 3:

| Time | Flow | Solvent composition | Solvent composition |
|---|---|---|---|
| [min] | [mL/min] | Eluent A [%] | Eluent B [%] |
| 0 | 1.0 | 100 | 0 |
| 7 | 1.0 | 62.5 | 37.5 |
| 10 | 1.0 | 62.5 | 37.5 |
| 26 | 1.0 | 58.5 | 41.5 |
| 31 | 1.0 | 58.5 | 41.5 |
| 33 | 1.0 | 0 | 100 |
| 35 | 1.0 | 0 | 100 |
| 37 | 1.0 | 100 | 0 |
| 45 | 1.0 | 100 | 0 |

Eluted species were detected and displayed on a graph showing the concentration of the eluted species vs. time. The elution profile showed a main peak with the unmodified protein and some further peaks eluting before and after the main peak, representing hydrophilic and hydrophobic variants of the protein, respectively. The total area of all peaks as well as the area of the single peaks was determined. Table 4 shows the percentage of the peak area for hydrophilic species in relation to the total peak area of the eluted species for the syringes of Table 1.

TABLE 4

| Condition | Syringe | Hydrophilic species (%) |
|---|---|---|
| T0 | S2 | 1.41 |
| | S3 | 1.21 |
| | S4 | 1.31 |
| | S5 | 1.33 |
| | S6 | 1.39 |
| 2 W 25° C. | S2 | 1.55 |
| | S3 | 1.49 |
| | S4 | 1.56 |
| | S5 | 1.53 |
| | S6 | 1.61 |
| 2 W 40° C. | S2 | 2.47 |
| | S3 | 1.89 |
| | S4 | 2.19 |
| | S5 | 2.47 |
| | S6 | 1.90 |
| 1 M 25° C. | S2 | 1.65 |
| | S3 | 1.63 |
| | S4 | 1.71 |
| | S5 | 1.75 |
| | S6 | 1.61 |
| 1 M 40° C. | S2 | 3.48 |
| | S3 | 3.30 |
| | S4 | 3.35 |
| | S5 | 4.35 |
| | S6 | 3.63 |
| 3 M 5° C. | S2 | 1.43 |
| | S3 | 2.42 |
| | S4 | 1.50 |
| | S5 | 1.67 |
| | S6 | 2.46 |
| 3 M 25° C. | S2 | 2.41 |
| | S3 | 2.28 |
| | S4 | 2.38 |
| | S5 | 2.56 |
| | S6 | 2.38 |
| 3 M 40° C. | S2 | 10.65 |
| | S3 | 6.12 |
| | S4 | 8.32 |
| | S5 | 12.41 |
| | S6 | 6.74 |

W: weeks;
M: months

### b) Cation Exchange Analysis

The protein samples from the syringes were loaded onto a Dionex, BioLCProPac® WCX-10, 4.0×250 mm, 10 μm column to detect acidic and basic variants of the protein.

The protein was eluted with a gradient of mobile phase A (20 mM potassium phosphate buffer, ph 6.0) and mobile phase B (250 mM KCl, 20 mM potassium phosphate buffer, ph 6.0) according to the following Table 5:

| Time | Solvent composition | Solvent composition |
|---|---|---|
| [min] | [%-B] | [mM KCl] |
| 0 | 0 | 0 |
| 3 | 0 | 0 |
| 33 | 50 | 125 |
| 35 | 50 | 125 |
| 36 | 0 | 0 |
| 40 | 0 | 0 |

Eluted species were detected and displayed on a graph showing the concentration of the eluted species vs. time. The elution profile showed a main peak with the unmodified protein and some further peaks eluting before and after the main peak, representing acidic and basic variants of the protein, respectively. The total area of all peaks as well as the area of the single peaks was determined. Table 6 shows the percentage of the peak area for acidic variants and basic variants, respectively, in relation to the total peak area of the eluted species for the syringes of Table 1.

TABLE 6

| | Syringe | Acidic species [%] | Basic Species [%] |
|---|---|---|---|
| T0 | S2 | 0.05 | 0.30 |
| | S3 | 0.04 | 0.31 |
| | S4 | 0.05 | 0.33 |
| | S5 | 0.05 | 0.38 |
| | S6 | 0.04 | 0.32 |
| 2 W 25° C. | S2 | 0.26 | 0.51 |
| | S3 | 0.15 | 0.54 |
| | S4 | 0.17 | 0.62 |
| | S5 | 0.22 | 0.67 |
| | S6 | 0.13 | 0.54 |
| 2 W 40° C. | S2 | 0.90 | 1.77 |
| | S3 | 0.75 | 1.69 |
| | S4 | 1.16 | 2.93 |
| | S5 | 1.30 | 3.20 |
| | S6 | 0.74 | 1.84 |
| 1 M 25° C. | S2 | 0.41 | 0.65 |
| | S3 | 0.30 | 0.72 |
| | S4 | 0.43 | 0.84 |
| | S5 | 0.56 | 2.12 |
| | S6 | 0.30 | 0.69 |
| 1 M 40° C. | S2 | 1.93 | 2.84 |
| | S3 | 2.19 | 4.51 |
| | S4 | 2.28 | 4.02 |
| | S5 | 2.86 | 5.50 |
| | S6 | 2.41 | 4.87 |
| 3 M 5° C. | S2 | 0.23 | 0.54 |
| | S3 | 0.15 | 0.49 |
| | S4 | 0.15 | 0.49 |
| | S5 | 0.12 | 0.49 |
| | S6 | 0.12 | 0.50 |
| 3 M 25° C. | S2 | 1.53 | 2.93 |
| | S3 | 1.10 | 2.67 |
| | S4 | 1.24 | 3.12 |
| | S5 | 1.31 | 3.46 |
| | S6 | 1.32 | 3.06 |
| 3 M 40° C. | S2 | 9.44 | 10.38 |
| | S3 | 6.47 | 7.86 |
| | S4 | 7.20 | 9.67 |
| | S5 | 9.89 | 13.31 |
| | S6 | 6.87 | 8.23 |

W: weeks;
M: months

### c) Size Exclusion Chromatography

The protein samples from the syringes were loaded onto a YMC-Pack Diol-200, 5 μm, 20 nm (8.0×300 mm) column to detect aggregates of the protein.

The protein was eluted by isocratic elution using 0.1 M potassium phosphate and 0.2 M sodium chloride. Eluted species were detected and displayed on a graph showing the concentration of the eluted species vs. time. The elution profile showed a main peak with the non-aggregated protein and some further peaks of the protein representing aggregated forms of the protein. The area of all peaks was determined. Table 7 shows the percentage of peak area for the aggregates in relation to the total peak area of the eluted species for the syringes of Table 1.

TABLE 7

| Condition | Syringe | Aggregates [%] |
|---|---|---|
| T0 | S2 | 0.04 |
| | S3 | 0.06 |
| | S4 | 0.05 |
| | S5 | 0.05 |
| | S6 | 0.06 |
| 2 W 25° C. | S2 | 0.10 |
| | S3 | 0.08 |
| | S4 | 0.06 |
| | S5 | 0.08 |
| | S6 | 0.06 |
| 2 W 40° C. | S2 | 0.14 |
| | S3 | 0.13 |
| | S4 | 0.12 |
| | S5 | 0.15 |
| | S6 | 0.11 |
| 1 M 25° C. | S2 | 0.08 |
| | S3 | 0.11 |
| | S4 | 0.09 |
| | S5 | 0.11 |
| | S6 | 0.08 |
| 1 M 40° C. | S2 | 0.24 |
| | S3 | 0.18 |
| | S4 | 0.18 |
| | S5 | 0.26 |
| | S6 | 0.19 |
| 3 M 5° C. | S2 | 0.06 |
| | S3 | 0.07 |
| | S4 | 0.07 |
| | S5 | 0.07 |
| | S6 | 0.06 |
| 3 M 25° C. | S2 | 0.18 |
| | S3 | 0.13 |
| | S4 | 0.12 |
| | S5 | 0.15 |
| | S6 | 0.12 |
| 3 M 40° C. | S2 | 0.90 |
| | S3 | 0.34 |
| | S4 | 0.44 |
| | S5 | 0.85 |
| | S6 | 0.48 |

From the results shown in Tables 2, 4, 6 and 7 it is apparent that the stability of ranibizumab in the pre-filled plastic syringe of the present invention (syringe 6) is at least comparable with the stability in the glass syringes under the conditions tested.

### 3. Determination of Gliding Forces in Different Syringes Containing Ranibizumab

The syringes as listed above in Table 1 were tested for their stopper movement forces, i.e. the break loose force and the gliding force. To this end, 400 μl of a solution containing 10 mM histidine buffer, 10% (w/v) trehalose dihydrate, 0.010% (w/v) polysorbate 20, pH 5.5, i.e. the components of the ranibizumab formulation, but not ranibizumab itself, were filled into the above syringes. Prior to testing, 27G×0.5″ needles were attached to the luer cone syringes. The testing was performed at a stopper speed of 190 mm/min over a travel length of 10.9 mm in a Tensile testing machine (TH2730, Thümler).

The results of the test are shown in Table 8 below.

TABLE 8

| | | Break loose force | |
|---|---|---|---|
| | | of syringes | Gliding Forces |
| Condition | Syringe | Average of 5 syringes | Average of 5 syringes |
| T0 | S2 | 3.3 | 4.7 |
| | S3 | 4.8 | 4.4 |
| | S4 | 3.9 | 3.3 |
| | S5 | 6.3 | 3.7 |
| | S6 | 2.2 | 4.9 |
| 1 M 5° C. | S2 | 3.9 | 4.9 |
| | S3 | 5.1 | 4.3 |
| | S4 | 4.6 | 3.5 |
| | S5 | 10.4 | 4.1 |
| | S6 | 5.0 | 4.8 |
| 1 M 25° C. | S2 | 3.7 | 4.2 |
| | S3 | 5.1 | 4.1 |
| | S4 | 4.9 | 3.6 |
| | S5 | 14.1 | 4.3 |
| | S6 | 4.6 | 4.6 |
| 1 M 40° C. | S2 | 4.4 | 4.1 |
| | S3 | 5.9 | 4.4 |
| | S4 | 5.0 | 4.1 |
| | S5 | 22.9 | 4.1 |
| | S6 | 4.5 | 4.4 |
| 3 M 5° C. | S2 | 5.8 | 5.2 |
| | S3 | 5.4 | 4.6 |
| | S4 | 5.0 | 5.5 |
| | S5 | 15.0 | 4.0 |
| | S6 | 2.6 | 5.2 |
| 3 M 25° C. | S2 | 6.4 | 5.5 |
| | S3 | 6.1 | 4.5 |
| | S4 | 5.3 | 4.1 |
| | S5 | 25.0 | 4.3 |
| | S6 | 2.9 | 5.2 |
| 3 M 40° C. | S2 | 6.2 | 5.4 |
| | S3 | 6.3 | 5.3 |
| | S4 | 5.6 | 4.9 |
| | S5 | 32.3 | 4.4 |
| | S6 | 3.0 | 5.2 |

The pre-filled silicone-free cycloolefin polymer syringe 6, i.e. the syringe of the present invention, has a gliding behavior which is comparable or even superior to that of the glass syringes which are coated with silicone oil.

### 4. Determination of Particles of Different Sizes in Different Syringes Containing Aflibercept and Subjected to Different Conditions

400 μl of a solution of the VEGF receptor fusion protein aflibercept containing 1 mg/ml of the antibody and 10 mM sodium phosphate buffer, 40 mM sodium chloride, 5% (w/v) sucrose, 0.03% (w/v) polysorbate 20, pH 6.2 was filled into the following syringes:

TABLE 9

| | | | | Silicone | |
|---|---|---|---|---|---|
| | Syringe | Syringe | Syringe | level | Stopper |
| No. | size | barrel | type | [mg] | coating |
| 2 | 1.0 ml | Borosilicate | Luer cone | Baked-on | Fluoropolymer |
| | | glass | | | (Fluorotec) |
| 3 | 1.0 ml | Borosilicate | Luer cone | 0.7 | Fluoropolymer |
| | | glass | | | (Fluorotec) |
| 4 | 1.0 ml | Borosilicate | Staked | 0.25 ± 0.2 | Fluoropolymer |
| | | glass | needle | | (Fluorotec) |
| 5 | 1.0 mL | Cycloolefin | Luer cone | 1.5 | Cross-linked |
| | | polymer | | | silicone |
| 6 | 1.0 mL | Cycloolefin | Luer cone | No silicone | Fluoropolymer |
| | | polymer | | | (Fluorotec) |

The syringes from Table 9 were rotated from needle to stopper with a speed of 1 cycle/10 seconds at 40° C. for five minutes, two weeks and four weeks or were subjected to five freeze/thaw cycles (+5 to −20° C. with 1° C./min). The syringes were also incubated at 5° C. for three, six and twelve months, at 25° C./60% relative humidity for two weeks, one month and three months and 40° C./75% relative humidity without rotation and then analyzed as described above for the syringes from Table 1.

### 5. Determination of Gliding Forces in Different Plastic and Glass Syringes Containing Aflibercept

The syringes as listed above in Table 9 were tested for their stopper movement forces, i.e. the break loose force and the gliding force. To this end, 0.165 ml of a solution containing 10 mM sodium phosphate buffer, 40 mM sodium chloride, 5% (w/v) sucrose, 0.03% (w/v) polysorbate 20, pH 6.2, i.e. the components of the aflibercept formulation, but not aflibercept itself, was filled into the above syringes of Table 9. Prior to testing, 30G×0.5″ needles were attached to the luer cone syringes. The testing was performed at a stopper speed of 190 mm/min over a travel length of 10.9 mm in a Tensile testing machine (TH2730, Thumler).

6. Determination of Particles of Different Sizes in Different Syringes Filled with Aflibercept and Subjected to Different Conditions

400 μl of a solution containing the target formulation of aflibercept (10 mM histidine buffer, 40 mM sodium chloride, 5% (w/v) sucrose, 0.03% (w/v) polysorbate 20, pH 6.2), but not aflibercept itself, was filled into the syringes as listed in Table 10.

The syringes as listed in Table 10 were incubated at 5° C., 25° C./60% relative humidity and 40° C./75% relative humidity for up to 3 months. Afterwards, the samples were analyzed for their stopper movement forces, i.e. the break loose force and the gliding force, and for subvisible particles determined by microfluidic imaging (MFI).

TABLE 10

| | | | | Silicone | |
|---|---|---|---|---|---|
| | Syringe | Syringe | Syringe | level | Stopper |
| No. | size | barrel | type | [mg] | coating |
| 2 | 1.0 ml | Borosilicate | Luer cone | 0.16 | Fluoropolymer |
| | | glass | | (baked-on) | (Flurotec) |
| 6 | 1.0 mL | Cycloolefin | Luer cone | No silicone | Fluoropolymer |
| | | polymer | | | (Flurotec) |

### a) Subvisible Particles Determined by MFI

The syringes from Table 10 were incubated at 5° C., 25° C./60% relative humidity and 40° C./75% relative humidity for three months. Afterwards, the light obscuration was determined with the FlowCam PV bench top system (Fluid Imaging Technologies Inc., Maine, USA) using the System software (VisualSpreadsheet software, version 3.4.8) and the following parameters:

| | |
|---|---|
| Mode: | AutoImage |
| Priming Method: | manual prime with sample |
| Flow Rate: | 0.100 ml/min |
| Recalibrations: | 0 |
| Stop Reason: | Sample Volume Processed |
| Sample Volume Aspirated: | 1.0421 ml |
| Sample Volume Processed: | 1.0392 ml |
| Fluid Volume Imaged: | 0.3421 ml |
| Frame Rate: | 22.00 fps |
| Magnification: | 10X |
| Calibration Factor: | 0.6979 |
| Syringe Size: | 1.00 ml |

5 syringes were pooled and measured at each time point. The syringes were emptied through the cone area and about 1 mL of the sample was measured undiluted. The results of the analysis are shown in Table 11. The pre-filled silicone-free cycloolefin polymer syringe 6 had low particle levels under all conditions tested whereas the baked-on siliconized glass syringe S2 comprised levels of particles ≥10 μm which miss the USP <789> specification for ophthalmic use when stored for 3 months at elevated temperatures.

TABLE 11

Particle count/mL [mean]

| Condition | Syringe | ≥10 μm | ≥25 μm |
|---|---|---|---|
| T0 | S2 | 21 | 2 |
| | S6 | 12 | 2 |
| 3 M 5° C. | S2 | 18 | 3 |
| | S6 | 20 | 1 |
| 3 M 25° C./ | S2 | 71 | 4 |
| 60% r.H. | S6 | 9 | 2 |
| 3 M 40° C./ | S2 | 169 | 5 |
| 75% r.H. | S6 | 15 | 1 |

M: months

7. Determination of Break Loose andGliding Forces in Plastic and Glass Syringe

The syringes from Table 10 were incubated at 5° C., 25° C./60% relative humidity and 40° C./75% relative humidity for one and three months. They all were filled with 0.400 mL of sterile filtered formulation and tested with regard to the break loose force and the gliding force of the syringe system.

5 samples were measured for each time point. The test was performed with a stopper speed of 190 mm/min covering a travel length of 100 mm in a Tensile testing machine (TH2730, Thümler).

Prior to the testing procedure 27 G×0.5″ needles were attached to the luer cone syringes S2 and S6.

TABLE 12

| | | Break | | Gliding | SD |
|---|---|---|---|---|---|
| | | loose force | SD Break | force | Gliding |
| Condition | Syringe | [Mean] | loose force | [Mean] | force |
| T0 | S2 | 4.2N | 0.4N | 4.8N | 0.2N |
| | S6 | 2.6N | 0.6N | 4.8N | 0.2N |
| 1 M 5° C. | S2 | 4.8N | 0.4N | 4.5N | 0.5N |
| | S6 | 2.2N | 0.1N | 3.6N | 0.1N |
| 3 M 5° C. | S2 | 4.8N | 0.5N | 5.1N | 0.2N |
| | S6 | 2.7N | 0.4N | 4.7N | 0.3N |
| 1 M 25° C./ | S2 | 4.6N | 0.7N | 4.1N | 1.0N |
| 60% r.H. | S6 | 2.3N | 0.2N | 3.8N | 0.2N |
| 3 M 25° C./ | S2 | 5.4N | 0.6N | 4.6N | 0.4N |
| 60% r.H. | S6 | 2.5N | 0.3N | 5.0N | 0.3N |
| 1 M 40° C./ | S2 | 5.2N | 0.3N | 4.5N | 0.6N |
| 75% r.H. | S6 | 2.2N | 0.2N | 4.1N | 0.2N |
| 3 M 40° C./ | S2 | 6.2N | 0.5N | 5.4N | 0.2N |
| 75% r.H. | S6 | 2.6N | 0.3N | 5.2N | 0.2N |

M: months

The pre-filled silicone-free cycloolefin polymer syringe 6, i.e. the syringe of the present invention, has a break loose and gliding behavior which is comparable or even superior to that of the glass syringes which are coated with silicone oil.

### 8. Determination of Aflibercept Stability in Plastic and Glass Syringes Subjected to Different Conditions

### a) Sample Preparation

165 μl of a solution containing 40 mg/ml of the VEGF antagonist aflibercept and 10 mM histidine buffer, 40 mM sodium chloride, 5% (w/v) sucrose, 0.03% (w/v) polysorbate 20, pH 6.2 was filled into the syringes as listed in Table 10 and the syringes were incubated at 5° C., 25° cl 60% relative humidity and 40° C./75% relative humidity for one month and 3 months.

Afterwards, the samples were analyzed by UV-Vis for protein concentration, by size exclusion chromatography (SEC) and asymmetric flow field-flow fractionation (AF4) for the presence of high molecular weight species (HMWS), by non-reduced sodium dodecyl sulfate polyacrylamide gel electrophoresis (SDS-PAGE) for the presence of fragments and HMWS, by reduced peptide mapping for the presence of methionine oxidation and deamidation. Isoelectric focusing (IEF) was used to analyze samples for chemical modifications which results in charge variants of aflibercept. Also pH was monitored within the whole incubation period.

During the complete stability program no significant change in protein concentration (spectrophotometric quantification at 280 nm; n=3) and pH (n=2) was detected in all samples.

### b) Af4

The asymmetric flow field flow fractionation (AF4) is a technique to identify and quantify higher molecular weight species of aflibercept based on their size. This separation is obtained by the difference in mobility (diffusion coefficient) in the flow field induced by the liquid flow across the channel. In combination with MALS (multi angle light scattering) and UV (280 nm) as concentration-dependent detector, the aflibercept aggregates can be characterized and quantified.

20 μg aflibercept were loaded onto a 15.5 cm separation channel 15.5 cm (short channel) combined with a W490 separation spacer (both Wyatt Technology) and a PLGC 10 kD SC-5 Membrane (Millipore). The protein was eluted using 0.1 M sodium phosphate (pH 6.0) and 0.02% sodium azide according to elution conditions shown in Table 13 representing the cross flow and focus flow during the separation (channel flow: 0.8 mL/min).

Eluted species were detected at a wavelength of 280 nm and displayed on a graph showing the concentration of the eluted species vs. time. The elution profile showed a main peak with the non-aggregated protein and some further peaks of the protein representing higher molecular weight forms of the protein. The corresponding molecular weights were calculated with a MALLS detector.

TABLE 13

| | | | | X_{Start} | X_{End} | FF |
|---|---|---|---|---|---|---|
| | Delta t | Time | | [mL/ | [mL/ | [mL/ |
| Step | [min] | [min] | Mode | min] | min] | min] |
| 1 | 4.0 | 4.0 | Elution | 1.5 | 1.5 | — |
| 2 | 1.0 | 5.0 | Focus | — | — | 2.0 |
| 3 | 2.0 | 7.0 | Focus + Inj. | — | — | 2.0 |
| 4 | 1.0 | 8.0 | Focus | — | — | 2.0 |
| 5 | 32.0 | 40.0 | Elution | 1.5 | 1.5 | — |
| 6 | 10.0 | 50.0 | Elution | 1.5 | 0.2 | — |
| 7 | 10.0 | 60.0 | Elution | 0.2 | 0.2 | — |
| 8 | 10.0 | 70.0 | Elution + Inj. | 0.2 | 0.0 | — |
| 9 | 10.0 | 80.0 | Elution + Inj. | 0.0 | 0.0 | — |

Table 14 shows the percentage of peak areas for the higher molecular weight species in relation to the total peak areas of the eluted species for the 1 and 3 months 40° C./75% relative humidity incubated syringes of Table 10. Each sample was examined in duplicate measurements unless otherwise noted.

All other temperatures (5° C. and 25° C./60% relative humidity) showed no significant increase of higher molecular weight species during storage compared to the starting material.

TABLE 14

| Condition | Syringe | HMWS [%] | SD [%] |
|---|---|---|---|
| T0 | S2 | 1.1 | n.a.*⁾ |
| | S6 | 1.1 | n.a.*⁾ |
| 1 M 40° C. | S2 | 10.7 | 0.1 |
| | S6 | 10.2 | 0.4 |
| 3 M 40° C. | S2 | 26.8 | 0.7 |
| | S6 | 26.3 | n.a.*⁾ |

*⁾only single measurement

The generation of HMWS determined by AF4-MALS was highly comparable during incubation at 40° C./75 relative humidity between the two syringes S2 (glass syringe) and S6 (COP) in the period up to 3 months. Both the identities of the higher molecular weight species and the temperature dependent kinetics were comparable between the two primary packaging systems.

### c) SEC

The protein samples from the syringes were loaded onto a TSKgel G3000SWXL, (Tosoh, 300×7.8 mm, 5 μm) column to detect high molecular weight species of aflibercept.

The protein was eluted by isocratic elution using 0.02 M sodium phosphate (pH 6.0) and 0.8 M sodium chloride at a flow rate of 1.0 mL/min at 25° C. Eluted species were detected at a wavelength of 214 nm and displayed on a graph showing the concentration of the eluted species vs. time. The elution profile showed a main peak with the non-aggregated protein and some further peaks of the protein representing higher molecular weight forms of the protein. The area of all peaks was determined.

Table 15 shows the percentage of peak area for the aggregates in relation to the total peak area of the eluted species for the syringes of Table 1. Each sample was examined in duplicate measurements.

TABLE 15

| Condition | Syringe | HMWS [%] | SD [%] |
|---|---|---|---|
| T0 | S2 | 2.20 | 0.01 |

| | | | | |
|---|---|---|---|---|
| | | S6 | 2.19 | 0.02 |
| 1 M | 5° C. | S2 | 2.31 | 0.01 |
| | | S6 | 2.26 | 0.01 |
| 3 M | 5° C. | S2 | 2.38 | 0.01 |
| | | S6 | 2.36 | 0.02 |
| 2 W | 25° C. | S2 | 2.45 | 0.01 |
| | | S6 | 2.45 | 0.00 |
| 1 M | 25° C. | S2 | 2.55 | 0.01 |
| | | S6 | 2.53 | 0.01 |
| 3 M | 25° C. | S2 | 3.03 | 0.01 |
| | | S6 | 3.01 | 0.00 |
| 0.5 M | 40° C. | S2 | 9.80 | 0.02 |
| | | S6 | 9.76 | 0.06 |
| 1 M | 40° C. | S2 | 15.58 | 0.01 |
| | | S6 | 15.49 | 0.06 |
| 3 M | 40° C. | S2 | 33.71 | 0.01 |
| | | S6 | 33.93 | 0.05 |

The generation of HMWS determined by SEC was highly comparable between the two syringes S2 (glass syringe) and S6 (COP) for all incubation parameters (temperature, storage time). Both the identities of the higher molecular weight species and the temperature dependent kinetics were comparable between the two primary packaging systems.

### d) Non-Reduced_SDS-PAGE

By non-reduced SDS-PAGE physical modifications such as fragmentation and oligomerization of aflibercept in the different syringe systems according to Table 10 were determined.

The SDS-PAGE was performed under non-reducing conditions in a 4-12% Tris-Glycine gel. Samples were pre-diluted to 0.4 mg/ml with water and further diluted to 0.2 mg/ml with SDS sample buffer. The samples were incubated at 95° C. for 5 min. After the run the gel was rinsed three times with 100 mL deionized water and dyed with Coomassie overnight at room temperature. After discoloration the gel was scanned and analyzed using QuantityOne Software. The running conditions were as follows:

| | |
|---|---|
| voltage: | 125 V |
| current: | 35 mA |
| power: | 5 W |
| time: | 130 min |

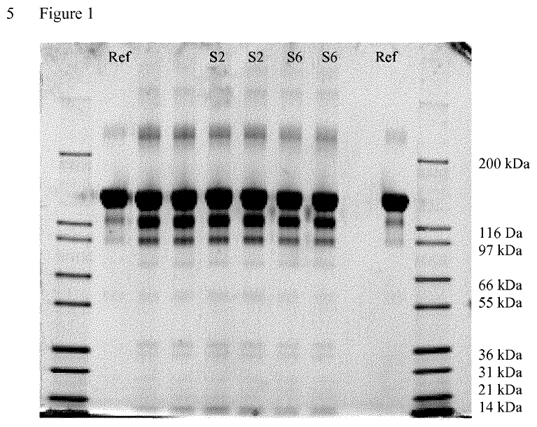

Non-reduced SDS-PAGE was performed for the samples at all temperatures during the complete incubation period of 3 months. Storing the samples at 5° C. did not lead to significant changes of the banding pattern in all primary packaging systems, no generation of new impurity bands or significant increment of existing impurity bands could be detected in both syringe materials over the whole incubation period. Storing the samples at 25° C./60% relative humidity led to stronger impurity bands, the results of the non-reduced SDS PAGE analysis of samples incubated for three months at 40° C./75% relative humidity are shown in FIG. 1.

In the non-reduced SDS-PAGE analysis of all samples incubated for three months at 40° C./75 relative humidity bands representing fragments and higher molecular weight species of aflibercept were visible. The generation of fragments and HMWS during the 3 months incubation was highly comparable as well in the kinetics and the identity of the impurities in both primary packaging systems shown in Table 10.

### e) Ief

Isoelectric focusing (IEF) separates different isoforms of aflibercept due to differences in their isoelectric points because of e.g. deamidation. The ready-to-use IEF gel (Focus Gel (pH 6-11) from Serva, No. 43329.01) contains a pH gradient within the gel. After application, proteins migrate due to their net charge in the pH gradient until they reach the pH equivalent to their isoelectric point (IEP, IP). Aflibercept samples were diluted to 0.5 mg/ml with ultrapure water. 10 μl thereof equal to 5 μg aflibercept were applied onto the focus gel. Each sample was analyzed as duplicate.

After the run the proteins were fixed for 60 minutes in a solution containing 12% (w/v) trichloroacetic acid and 3.5% 5-sulfosalicyl acid dihydrate (w/v), rinsed three times with deionized water and dyed with Coomassie overnight at room temperature. After discoloration with 20% ethanol the gel was scanned with a GS 800 densitometer from BioRad and analyzed.

Table 16 shows the focusing conditions:

TABLE 16

| | Time | Power | Current | Voltage |
|---|---|---|---|---|
| Phase | (min) | (W) | (mA) | (V) |
| Pre focusing | 20 | 10 | 50 | 1000 |
| Sample entrance | 30 | 10 | 30 | 500 |
| Isoelectric focusing | 90 | 20 | 18 | 1500 |
| Sharpening | 30 | 25 | 15 | 2000 |

In the IEF no change in the banding pattern of aflibercept compared to the reference could be detected in all primary packaging systems after one month storage at all temperatures. After 3 months only the samples incubated at 5° C. and 25° C./60% complied with the reference and showed no alteration in comparison to the starting material. Samples incubated at 40° C./75% relative humidity comprised a comparable shift to acidic species in all tested primary packaging materials, so that there was no difference with regard to the different primary packaging materials shown in Table 10.

### f) Reduced Peptide Mapping

By reduced peptide mapping the purity of aflibercept with regard to deamidation and methionine oxidation was analyzed after digestion with trypsin and liquid chromatography coupled to mass spectrometry (LC-MS)

After reduction and alkylation, the protein was submitted to enzymatic cleavage with trypsin. The resulting peptides were analyzed by RP-UPLC-MS. During chromatography the peptides were eluted by changing the mobile phase from highly polar (trifluoroacetic acid in water) to less polar (trifluoroacetic acid in acetonitrile) and analyzed by mass spectrometry (Xevo G2-XS QTOF). The peptide data was processed and compared with the theoretical protein sequence and a reference sample to detect oxidations and deamidations.

The syringes shown in Table 10 were analyzed as single measurement after 3 months incubation at 5° C., 25° C./60% relative humidity and 40° C./75 relative humidity and compared to the starting material t0.

Samples were diluted with denaturation buffer (50 mM Tris(hydroxymethyl)amino-methane) to a aflibercept concentration of 1.25 mg/mL. 80 μl of the diluted samples were mixed with 10 μl of 0.5% RapiGest (from Waters, solved in 50 mM Tris-(hydroxymethyl)aminomethane) and incubated 5 minutes at 95° C. 4.5 μl of 0.02 M DTT (solved in 50 mM Tris(hydroxymethyl)-aminomethane) were added for reduction and incubated for 30 minutes at 37° C. For aflibercept digestion 5 μl of a 1 mg/mL Trypsin solution (solved in 50 mM acetic acid) were added and incubated for further 3 hours at 37° C. The reaction was stopped with 20 μl of 2% (v/v) trifluoro-acetic acid and an incubation for 30 minutes at 37° C. The supernatant was diluted to 0.125 mg/mL with 50 mM Tris(hydroxymethyl)-aminomethane for analysis of the peptides.

### Uplc Parameters:

The digested protein samples from the syringes were loaded onto an ACQUITY UPLC-CSH C-18 column from Waters, 100 mm×2.1 mm, 1.7 μm. 0.25 μg of the digested samples were eluted at 65° C. with a gradient of eluent A (water), eluent B (acetonitrile), eluent C (0.25% trifluoroacetic acid) and D (n-propanol) according to the following Table 17:

TABLE 17

| Time | Eluent A | Eluent B | Eluent C | Eluent D |
|---|---|---|---|---|
| [minutes] | [%] | [%] | [%] | [%] |
| 0.0 | 89.0 | 1.0 | 10.0 | 0.0 |
| 2.5 | 89.0 | 1.0 | 10.0 | 0.0 |
| 5.0 | 80.0 | 8.0 | 10.0 | 2.0 |
| 50.0 | 57.5 | 26.0 | 10.0 | 6.5 |
| 52.0 | 0.0 | 72.0 | 10.0 | 18.0 |
| 54.0 | 0.0 | 72.0 | 10.0 | 18.0 |
| 56.0 | 89.0 | 1.0 | 10.0 | 0.0 |
| 60.0 | 89.0 | 1.0 | 10.0 | 0.0 |

Method parameters for mass spectrometry:

| Ionisation type: | ESI | | Polarity: | Positive | |
|---|---|---|---|---|---|
| Analyser mode: | Sensitivity | | Experiment type: | MS | |
| Start Mass: | 50 | m/z | Cone Gas Flow: | 30 | L/h |
| End Mass: | 2000 | m/z | Desolvation Gas Flow: | 1000 | L/h |
| Source Temperature: | 120° | C. | Scan Time: | 0.5 | s |
| Desolvation | 450° | C. | Capillary Voltage: | 3.0 | kV |
| Temperature: | | | | | |
| Cone Voltage: | 35 | V | | | |

LockSpray Profile

| Reference Compound: | Leucine Enkephalin | |
|---|---|---|
| MS Lock mass: | 556.2766 | m/z |
| Scan Time: | 0.5 | s |
| Interval: | 30 | s |

4 oxidated methionines in aflibercept could be identified in the peptides (1:T1_AS20, 1:T22, 1:T28, 1:T48) and were summed up for evaluation of the total oxidation (see Table 18)

6 deamidations of aflibercept could be identified in the peptides (1:T10_AS12; 1:T11; 1:T10_AS12; 1:T12_AS3; 1:T12_AS3; 1:T30_AS12; 1:T30_AS?; 1:T33_AS14) and were summed up for evaluation of the total deamidation (see Table 18)

TABLE 18

| | | Total methionine | Total |
|---|---|---|---|
| Condition | Syringe | oxidations [%] | deamidations [%] |
| T0 | S2 | 23.1 | 35.3 |
| | S6 | 22.5 | 37.6 |
| 3 M 5° C. | S2 | 27.7 | 36.8 |
| | S6 | 22.4 | 36.9 |
| 3 M 25° C. | S2 | 24.5 | 44.0 |
| | S6 | 23.4 | 45.3 |
| 3 M 40° C. | S2 | 25.7 | 92.0 |
| | S6 | 27.3 | 90.0 |

Both syringes shown in Table 10 comprise an identical stability with regard to methionine oxidation and deamidation.

Whereas in all temperature conditions no significant increase of methionine oxidation could be detected in both syringe materials (glass vs. COP), the increase of deamidation was temperature dependent. Both syringe systems comprised a comparable increase of deamidation in the stability program.

From the results shown it is apparent that the stability of aflibercept in the pre-filled plastic syringe of the present invention (syringe 6) is at least comparable with the stability in the glass syringes (syringe 2) under the conditions tested.

1. Pre-filled syringe containing a liquid formulation of a VEGF antagonist and comprising a syringe barrel, wherein the syringe barrel is made of plastic and is silicone-free, and further comprising a non-retractable stopper.

2. Pre-filled syringe containing a liquid formulation of a VEGF antagonist and comprising a syringe barrel, wherein the syringe barrel is made of plastic, is silicone-free and has a length of 45 mm to 65 mm.

3. Pre-filled syringe according to claim 1 or 2, wherein the VEGF antagonist is an anti-VEGF antibody or an antigen-binding fragment of such antibody or a VEGF receptor fusion protein.

4. Pre-filled syringe according to claim 3, wherein the anti-VEGF antagonist is ranibizumab or aflibercept.

5. Pre-filled syringe according to any one of the preceding claims, wherein the antagonist concentration is 1 to 100 mg/ml.

6. Pre-filled syringe according to any one of the preceding claims, containing less than 50 particles per ml of the liquid formulation having a diameter of 10 μm or greater.

7. Pre-filled syringe according to any one of the preceding claims, containing less than 5 particles per ml of the liquid formulation having a diameter of 25 μm or greater.

8. Pre-filled syringe according to any one of the preceding claims, having a sliding force of less than or equal to 10N.

9. Pre-filled syringe according to any one of the preceding claims, further comprising a silicone-free stopper.

10. Pre-filled syringe according to any one of the preceding claims, wherein the syringe barrel is made of cycloolefin polymer or cycloolefin copolymer.

11. Pre-filled syringe according to any one of the preceding claims, wherein the syringe barrel comprises an internal coating other than a silicone coating.

12. Pre-filled syringe according to any one of the preceding claims, comprising a staked needle.

13. Kit comprising one or more pre-filled syringes of any one of the preceding claims.

14. Pre-filled syringe of any of claims 1 to 11 for use in administering a liquid formulation of a VEGF antagonist to a patient having an ocular disease.

15. Pre-filled syringe for the use according to claim 13, wherein the ocular disease is selected from the group consisting of age-related macular degeneration (AMD), visual impairment due to diabetic macular oedema (DME), visual impairment due to macular oedema secondary to retinal vein occlusion (branch RVO or central RVO), diabetic retinopathy in patients with diabetic macular edema or visual impairment due to choroidal neovascularisation (CNV) secondary to pathologic myopia.

16. Pre-filled syringe for the use according to claim 14 or 15, wherein a volume of 30 to 100 μl of the liquid formulation is administered to the patient.
